(19) United States
(12) Patent Application Publication
(10) Pub. No.: US 2010/0094420 A1
(43) Pub. Date: Apr. 15, 2010

(54) Porous Bone Fixation Device

(76) Inventor: Joseph A. Grohowski, JR., Glens Falls, NY (US)

Correspondence Address: Zitzmann Consulting, LLC;c/o CPA Global, P.O. Box 52050, Minneapolis, MN, 55402, US

(21) Appl. No.: 12/635,520

(22) Filed: Dec. 10, 2009

Related U.S. Application Data
(63) Continuation-in-part of application No. 11/450,059, filed on Jun. 9, 2006.
Application No. 11/450,059 is a continuation-in-part of application No. 10/884,444, filed on Jul. 2, 2004.

(51) Int. Cl. A61F 2/28; A61B 17/80

(52) U.S. Cl. 623/16.11; 606/289

(57) ABSTRACT

A porous bone fixation device has a body including non-porous areas that provide superior long term fixation and osteointegration performance. The porous areas provide an in-growth surface in strategic sections such as the threaded section of the fixation device that allow substantially more contact area between in-growth surface and bone. The in-growth material is a network of interconnected porosity in a matrix of bio compatible, preferably titanium. Examples of bone fixation devices include bone screws, bone anchors, dental implants, and similar devices used to provide a fixation point in bone.

### CROSS REFERENCE TO RELATED APPLICATIONS

This application is a continuation-in-part of U.S. patent application Ser. No. 11/450,059, which is a continuation-in-part of U.S. patent application Ser. No. 10/884,444, the entire specification of each of which is hereby incorporated by reference herein for all purposes.

### BACKGROUND OF THE INVENTION

1. Field of the Invention

This invention relates to bone fixation devices having a specified porosity and a method of manufacturing such articles by using an extractable particulate.

2. Description of the Related Art

Porous metal articles are used in many applications including orthopedic implants, bone growth media, bone fixation devices, filters, sound suppression materials, fuel cells, catalyst supports, and magnetic shielding. Bone fixation devices are used in many surgical procedures, often for fixation of implanted devices or for repair of bone fractures. They may also be used to fasten transcutaneous devices to bone. In many cases, the intent is to leave the fixation device in the body permanently. This may be because the fixation device continues to serve its purpose indefinitely or because it is simply more convenient and poses no threat to the patient.

Using bone fixation devices that include a porous in-growth surface may be preferred in certain orthopedic application to promote bone in-growth. Non-porous bone fixation devices can result in complications due to fatigue failure, loosening, and “windshield wiper” or toggling effect. Fatigue failure is a problem seen in several different clinical situations. Often conventional bone fixation devices simply wear unexpectedly quickly due to a particular clinical situation; in these cases, fixation devices most often break at the neck of the fixation device. Backing out or loosening can be due to issues such as installation, loading mechanics and poor bone apposition or poor bone quality. Micro-motion at the fixation device/bone interface can cause device failure.

Additionally, when bone fixation devices are used as part of a dynamic stabilization system, the fixation device is subjected to cyclic loading throughout the life of the device in more extreme ways. Because conventional fixation devices are simply pressing up against the bone and there in no actual integration of bone into the device, conventional fixation devices can loosen over time, creating a toggling effect visible as osteolysis around the fixation device on a diagnostic x-ray.

Conventional fixation devices may have limited clinical success due to the quality of the bone in which they are installed. Osteoporotic patient have significantly diminished bone density and consequently lower bone strength, which make the securing of a fixation device more difficult. Fixation devices in these patients can loosen much more easily, leading to device failure.

An uneven pore distribution throughout the porous metal article can affect the resulting physical properties of the porous metal article. This is of particular concern for porous bone fixation devices such as screws that may have threads with thin cross-sectional areas. Any reduction in the performance characteristics of the porous metal article can result in a failure of the bone fixation device.

Although highly desirable, there are inherent problems in the concept of a bone fixation device with a porous thread form. Many successful bone thread forms use threads having a sharp crest edge. The nature of porous material provides an uneven surface, which is aggravated as two porous planes converge at an edge to form a sharp thread. The porous edge of a thread has less strength and may be susceptible to breakage or chipping. Thus, a fixation device is desired having a tailored pore character with sufficient strength for bone in-growth applications

### SUMMARY OF THE INVENTION

The present invention provides a bone fixation device which is at least partially porous and exhibits a predetermined porosity defined by an extractable material. In one embodiment the device is a bone screw having a porous thread that can have a crest, and in another embodiment, the threads can have flat lands.

In one form thereof, this invention provides a bone fixation device having threads that are partially porous and have a shear strength of greater than 20 Megapascal. In one embodiment the device is a bone screw having a porous thread that can have a crest, and in another embodiment, the threads can have flat lands.

In still a further aspect, this invention provides a bone fixation device having threads that are partially porous and have a fatigue life of greater than 10,000,000 cycles. In one embodiment the device is a bone screw having a porous thread that can have a crest, and in another embodiment, the threads can have flat lands.

In still a further aspect, this invention provides bone fixation device and a method of manufacture of bone fixation device having a porous surface suitable for use as an implant device.

According to another aspect of the present invention, a method of manufacturing a porous bone fixation device is provided. The method comprises the steps of blending an extractable particulate and a first powder of metal particles to form a homogeneous mixture; forming an article comprising the mixture; providing the article in a mold; injecting a feedstock into the mold comprising a second powder of metal particles to form a composite article; removing the extractable particulate to form pores in the composite article; and sintering the composite article. In one embodiment of the method, the step of removing the extractable particulate comprises exposing the composite article to a water bath. In still another embodiment, the step of removing the extractable particulate also removes a binder from the feedstock.

### BRIEF DESCRIPTION OF THE DRAWINGS

FIG. 1 is a schematic of a compacted mixture illustrating the compacted matrix phase and the extractable phase.

FIG. 2 is a schematic of a presintered compact with the extractable phase partially extracted.

FIG. 3 is a schematic of a presintered compact with the extractable phase completely extracted.

FIG. 4 represents a micrograph at six times magnification showing a cross-section of a layered titanium material made in accordance with the present invention.

FIG. 5 is a side view of a typical implant in the upper femur.

FIG. 6 is a magnified view of the femur in FIG. 5 and shows in detail the interface between the implant and the bone.

FIG. 7 represents a micrograph at six times magnification showing the surface of porous titanium material made in accordance with the present invention.

FIG. 8 is a schematic describing an embodiment of forming a porous fixation device by filling a cold isostatic pressing tool with metal powder and pore-former.

FIG. 9 is a schematic describing an embodiment of forming a porous fixation device by sealing and applying isostatic pressure to the tool of FIG. 8

FIG. 10 is an embodiment of a green article in the shape of a cylinder comprising metal powder and pore former after the application of isostatic pressure as shown in FIG. 9.

FIG. 11 is a schematic describing an embodiment of forming a porous fixation device by machining the green cylinder of FIG. 10 on a lathe.

FIG. 12 is the embodiment of the cylinder of FIG. 10 after machining.

FIG. 13 is a schematic describing an embodiment of forming a porous fixation device by placing the machined cylinder of FIG. 12 in an injection molding tool.

FIG. 14 is an embodiment of a bone fixation device after injection molding.

FIG. 15 is a schematic describing an embodiment of forming a porous fixation device by green machining the section formed by the metal powder and pore former of the article of FIG. 14.

FIG. 16 is a perspective view of one embodiment of a porous bone fixation device.

FIG. 17 is a perspective view of another embodiment of a porous bone fixation device having porous threads and flat lands at the major diameter.

FIG. 18 is a partial cross-section of the embodiment of the bone fixation device of FIG. 17 revealing the interface of the porous and nonporous sections.

FIG. 19 is a perspective view of another embodiment of a porous bone fixation device having a porous thread and a crest thread edge at the major diameter.

FIG. 20 is a partial cross-section of the embodiment of a bone fixation device of FIG. 19 revealing the interface of the porous and nonporous sections.

FIG. 21 is a perspective view of another embodiment of a porous bone fixation device having porous threads and flat lands at the major diameter and a thread element having a non-porous section.

FIG. 22 is a partial cross-section of the embodiment of the bone fixation device of FIG. 21 revealing the interface of the porous and nonporous sections.

FIG. 23 is a perspective view of another embodiment of a porous bone fixation device having porous threads and flat lands at the major diameter.

FIG. 24 is a partial cross-section of the embodiment of the bone fixation device of FIG. 23 revealing the interface of the porous and nonporous sections and the tapering of the porous minor diameter.

FIG. 25 is a perspective view of another embodiment of a porous bone fixation device having porous threads and flat lands at the major diameter.

FIG. 26 is a partial cross-section of the embodiment of the bone fixation device of FIG. 25 revealing the interface of the porous and nonporous sections and having a tapered minor diameter.

### DESCRIPTION OF THE INVENTION

The present invention relates to porous metal articles having porosity characteristics that are determined by an extractable material which is removed prior to a final sintering and methods to manufacture such porous metal articles.

Powder metallurgy processes are used to form metal articles wherein a portion of the powder being processed is replaced with a pore forming material which is removed to form the desired porosity. The metal articles include articles that are elemental metal, metal alloys, or metal composites. The pore forming material is referred to as an extractable particulate or pore-former.

The powder and pore-former are mixed, the article is formed, optionally presintered and, thereafter, the pore-former is extracted. The powder remains to form the metal matrix of material around the pores formed by the extractable particulate. The matrix can then be further shaped and finally sintered to give the article greater strength.

The use of an extractable particulate to form porosity in an article formed from powder allows control over the pore properties, including pore density, size, distribution, and shape. The pore properties in the final article are determined primarily by the properties of the extractable particulate and, thereby, are tailored by the selection of the extractable particulate. By specifying one or more properties and processing the extractable particulate to reflect the desired pore properties, the present invention allows for the tailoring of the pore character.

The concentration or loading of the extractable particulate in the matrix determines the porosity density in the finished article. The extent of interconnectivity between the pores is varied by the concentration of the extractable particulate as well as the size of the pore forming material and the matrix forming powder. For example, a compact made using a titanium powder of a −325 mesh as the matrix material and 70 percent or more by volume potassium chloride granules having a mesh size of −20 to +60 will exhibit continuous interconnectivity. If the potassium chloride granules are reduced in size, interconnectivity will occur at a lesser volume fraction of porosity.

The required interconnectivity and concentration of extractable material used is dictated by the application. Some applications, such as osteointegration and filtration require pore interconnectivity throughout the article. Other applications such as those targeting a reduced weight or density may require a closed pore cell structure.

The difference in volume percent porosity of the initial compacted article compared with the sintered article depends on the materials and processing, but is generally less than ten percent. Changes in the percent porosity of a compact during processing are due to several factors. The primary factor is that the initial mixing of powders assumes a final density of 100% for the sintered matrix material, depending on the subsequent processing, sintered densities may vary from 85% to 100% dense. Applications desiring a more porous structure use a larger fraction of extractable material and applications desiring a less porous structure use a lesser fraction of extractable phase. Too high of a content of extractable material may render the compacted article too fragile to handle between extraction and sintering, or may inhibit compaction of the matrix material. Preferably, the pore former content will be between 50 and 90 percent by volume of the mixture. If a lower porosity content is desired, it may be advantageous to compact the material to relatively low density in order to allow the easy extraction of the pore former. Once the change in volume percent porosity for a particular combination of matrix powder and extractable material has been determined, it remains constant and thus permits the precise tailoring of porosity volume in the final article.

The pore structure and properties of the final article are tailored by the size and morphology of the extractable particulate. The size and shape properties of the extractable particulate depend on the material used as the extractable particulate. The shape of a pore (spherical, angular or irregular, etc.) will be determined by the extractable particulate used to form it. The size of a specific pore in the final article is proportional to the size of the extractable particulate that is used to form the pore. The proportionality is determined by any shrinkage that may result in the sintering of the metal powder material. The shrinkage encountered during sintering is dictated by the degree to which the matrix is densified. The shrinkage of an article during sintering can be approximated by the following formula:

ΔL/Lᵢ=1−(ρᵢ/ρ_{f})^{1/3}

Where:
ΔL=Change in length.
Lᵢ=Initial length.
ρᵢ=Initial density of matrix.
ρ_{f}=Final density of matrix.

This formula does not address changes in shrinkage due to changes in the chemistry of the matrix material such as carbon loss or oxygen pick-up. These are typically slight discrepancies and the actual results are generally within a few percent of the calculated results. Moreover, once the actual shrinkage has been determined for a specific matrix material, the invention provides for a very consistent and repeatable process with essentially no variability in shrinkage. This allows for precise control over and tailorability of the final pore size and shape.

The desired pore size distribution for the final article is determined by the particle size distribution of the extractable particulate. Although some shrinkage may be encountered during processing, the final pore size distribution is directly proportional to the particle size distribution of the extractable particulate. This proportionality is determined by the shrink percentage as discussed above. This relationship allows for the tailoring of articles with very specific pore size distribution by engineering the particle size distribution of the extractable particulate. Methods of tailoring particle character or size distributions are well known and include milling, grinding, sieving, and air classification. Pore size distributions can be manipulated to produce wide or narrow pore size distributions, as well a bi-modal or multi-modal distributions.

There are several means of obtaining desired pore former characteristics, including sieving a desired size range from a commercially available product, or manufacturing material specifically to the desired characteristics. This may also be accomplished by other conventional methods.

In addition to considerations related to the predetermination of the desired porosity, there are several process related considerations to be made when selecting an extractable particulate. In a preferred embodiment, the extractable particulate should be compatible with any additional processing steps, it should not leave undesired residues in the final part, it should not react with the matrix material, and it should have adequate strength so as not to be deformed during processing, such as compaction. Since the forming route includes elevated temperatures prior to particle extraction, the extractable particulate should exhibit stability at those temperatures.

The extractable particulate material should be removable by extraction via a fluid prior to finally sintering. In a preferred embodiment, the processing involves extraction through a water-based system. In this system, ionic bonded materials such as metal salts are desirable as extractable particulates because of ease of removal. Any salt ions should also be completely removed from the metal matrix prior to finally sintering.

Mechanical stability of the extractable article is important. In a preferred embodiment, the extractable particulate material should be selected so it will not store energy during any processing steps (for example, compaction) of the metal powder and extractable particulate mixture. If a material is compressed during the compaction process, then upon removal of the compaction force, the energy stored in the compacted particulate acts upon the strength of the compact. This may result in the compact cracking upon removal from the compaction tool or disintegrating during the water extraction step.

A variety of processing options may be additionally employed when forming the porous metal articles. For example, to enhance mechanical stability of the resulting compact, the mixture may optionally be presintered to some degree as described further herein. In this manner, the thermal stability of the extractable particulate may be taken advantage of while the tensile strength of the forming matrix is increased. As also described further herein, articles may be machined prior to removal of the extractable particulate. In this manner, pores at the surface of the articles are maintained in a relatively open state by the particulate during the machining. This helps avoid the undesired effect of physically isolating the porosity of the article. Further such process additions and modifications may similarly be employed, not limited to these particular examples.

According to the present invention, the extractable particulates are preferably salts, and most preferably potassium chloride, potassium sorbate, sodium chloride or a mixture thereof.

In one example for medical implant devices, angular titanium powder of a mesh size between −100 to +635 is be used as the matrix material with potassium chloride particles between 100 and 2000 micron as the extractable particulate.

In another example, potassium chloride is used as the extractable particulate. Granules of potassium sorbate that are between 600 and 1000 micron in diameter are well suited for use with an alcohol, ketone or water-soluble stainless steel feedstock system for use in metal injection molding.

The metal powder used should be chosen based on the desired properties of the final product. According the to present invention, the specification of the metal powder must be selected to ensure the article performs predictably during processing. Particles having an irregular, angular, or ligamental nature will deform around or into one another during processing, resulting in better green strength, i.e., the strength of the article after forming the metal powder and extractable material into a shape. For example, when using a compaction technique to form the shape, selecting metal powders with angular characteristics will give the compact adequate green strength. Hydride/dehydride processes for making metal powder result in a desirable angular structure suitable for formation by compaction. The metal powder is preferably titanium, tantalum, cobalt chrome, niobium, stainless steel, nickel, copper, aluminum, or any alloys thereof.

Once the extractable material and metal powder have been selected, the materials are then blended. Blending techniques such as V-blending, mixing on a jar mill, hand blending or use of other known powder mixing techniques can be used.

When the metal powder and the pore-forming material are blended together care must be taken to ensure that the materials form a homogenous mixture irrespective of any variation in particle size and density. In one embodiment, a small amount of a homogenizing aid can be added to help the different materials adhere to each other and create a homogenous mixture. In a preferred embodiment, the homogenization aid can easily be removed after the metal powder and pore-former are compacted. In a most preferred embodiment, the homogenizing aid either (a) is removed by the same fluid used to extract the extractable particulate, or (b) is stable at room temperature and pressure, but will evaporate at elevated temperatures or reduced pressure. Examples of such homogenizing aids include polyethylene glycol (PEG) and alcohols or isoparaffinic solvents, as well as organic liquids, such as acetone, which can easily be removed by evaporation prior to or after compaction.

In one example, titanium powder of a mesh size between −170 to +635 is used as the matrix material, with potassium chloride particles between 100 and 2000 micron, and eight percent by weight of acetone is added to the blend, resulting in a homogeneous mixture.

In order to ensure good blending and prevent agglomeration, the mixture may be sieved between blending steps or after blending. As another example, in a system using potassium chloride granules having a size range between 250 and 850 micron and a −325 mesh titanium powder and eight weight percent acetone the mixture can be sieved through a 1000 micron screen to ensure there are no significant agglomerates.

The homogenizing aid added to the blend to increase homogeneity can be selected to be easily removed before or after the compaction step. This is an important consideration and there are advantages to either approach. The inclusion of a third material to wet the pore-former and the matrix material can vastly improve homogeneity. If this homogenizing aid is left in the blended materials during the compaction, it can limit the degree to which the materials are compacted because it creates a hydrostatic lock at the limit of the mixture's compatibility. This results in the compacted matrix being slightly less dense than it would have been if the material was not present. In many cases this difference in density is not critical. If however, the highest possible compacted density is required then the homogenizing aid should be removed prior to compaction.

If the homogenization aid remains in the blend during the compacting step, the amount of material used as an aid should be optimized to minimize its effect on compaction while still providing adequate homogenization. In a preferred embodiment, polyethylene glycol (PEG) having a molecular weight of 200 is used to increase homogeneity and is removed after compaction during the water immersion step used to remove the extractable particulate. The amount of PEG 200 used is based on the theoretical density of the mixture of extractable and matrix material if there were no voids present. As an example, a porous titanium implantable device can be formed using potassium chloride granules having a size range between 250 and 850 micron with a −325 mesh titanium powder and 0.05 grams PEG 200 per cubic centimeter of the mixture at theoretical density.

After blending, the metal powder, extractable material, and any homogenizing aid is processed to achieve a desired green shape. This can be achieved by known powder consolidation techniques such as die compaction, isostatic pressing, or metal injection molding.

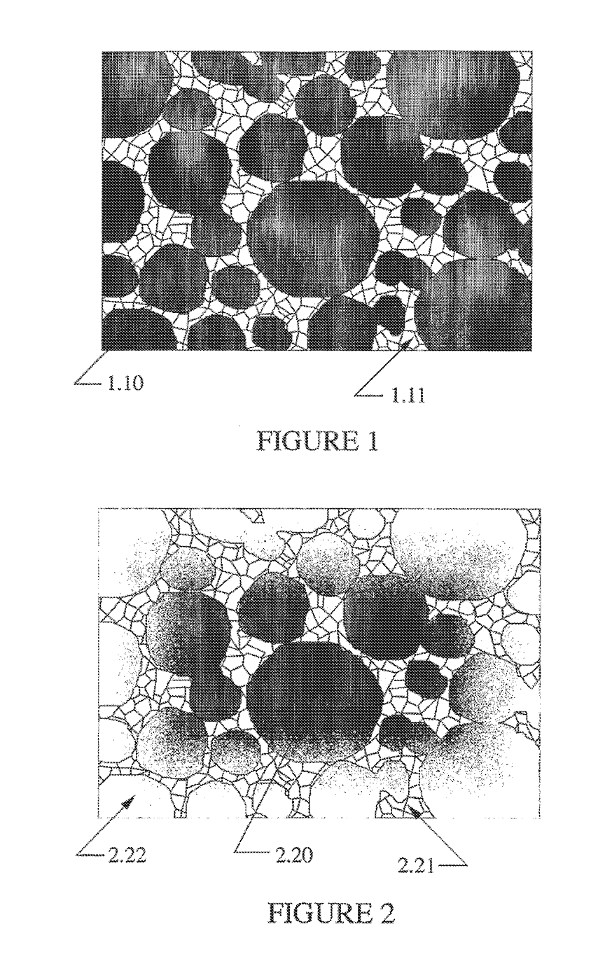

In one embodiment, the extractable particulate and the matrix powder are blended together and consolidated by compacting the mixture into a cylindrical shape. FIG. 1 shows a enlarged schematic of an extractable phase 10, and an angular matrix powder 11 after die compaction.

After the article is formed and optionally presintered, according to the present invention, the extractable particulate is dissolved out of the article by subjecting the article to an extraction process using a fluid. Dissolving the pore-former in a fluid in which the pore-former is soluble will remove the pore-former from the article. In a preferred embodiment, a compacted and optionally presintered article using a salt as a pore-former is immersed in water, thereby dissolving and removing the extractable particles. Other fluids, including gasses and liquids may be used depending on the dissolution properties of the extractable pore-former.

When using water to dissolve the extractable particulate, the water bath should be deoxygenated to reduce or eliminate the potential for oxidation of the metal powder in the water. This can be done by, for example, bubbling nitrogen through the water, or other known deoxygenating means.

FIG. 2 shows a schematic of a compacted mixture in accordance with the present invention with the pore-former partially extracted. The matrix powder 21 surrounds the remaining extractable phase 20 and the pores 22 formed during the extraction process. The pores 22 retain the dimensional properties of the extractable particulate used to form them.

By further using a rinsing step after immersion, any amount of pore-former remaining after exposure to the dissolution fluid, can be eliminated or reduced to negligible trace amounts. While certain metal systems may be less sensitive to contamination than others, it is generally desirable to reduce any extractable material remaining in the compacted article to below 2000 parts per million (ppm), and in applications such as medical implants, preferably to below 500 ppm, and most preferably below 200 ppm. As an example, a titanium porous article is made using potassium chloride as the pore-former and has a residual chlorine content in the green article of less than 25 ppm and a potassium content of less than 50 ppm. Such articles will have no significant contamination as a result of the pore-former and are suitable for orthopedic implant applications.

The amount of pore-former in the extraction fluid can be monitored by known means for analyzing the fluid. As an example, if water is used as the extraction fluid, the amount of extractable material in the water can be measured in ppm by a conductivity meter. Reduction on weight of the article can also be used to monitor removal of the extractable phase.

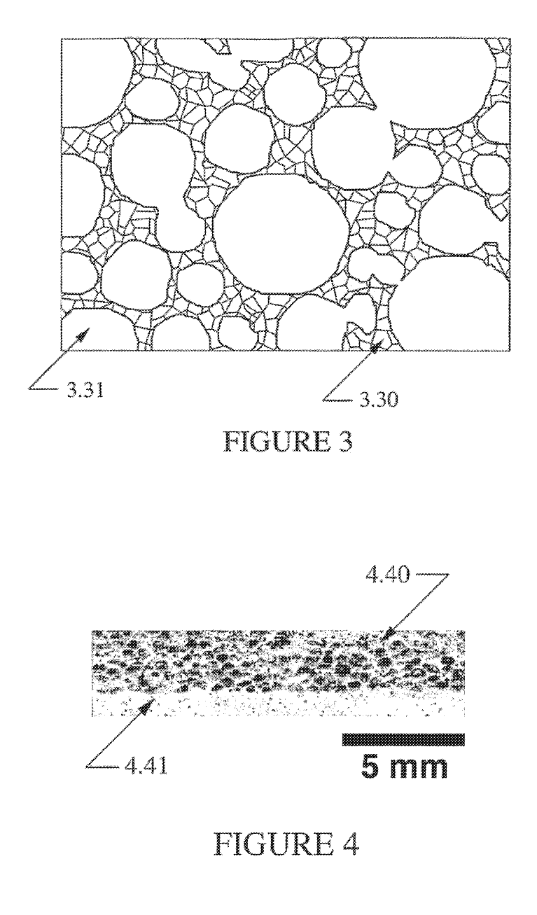

FIG. 3 represents a schematic of an article where the extractable phase has been removed. The compacted metal matrix 30 contains the porous region 31 formed by the removal of the extractable particulate. The article has adequate strength to allow handling.

As described above, the compact may be presintered prior to removing the extractable phase. In this manner, sinter bonds may be formed during the thermal cycle adding to the strength of the compact, apparent on removal of the extractable phase. It is important to presinter the compact at a temperature high enough to develop sinter bonds that impart strength. It is also important to stay below a thermal condition that would allow for later stage sintering, such as pore closure of the matrix material. If the pores are allowed to close before the extractable particulate is removed, the closed pores will prevent complete removal of the extractable particulate, resulting in contamination of the final product.

The extraction of the pore-forming material prior to the article being exposed to final sintering temperatures reduces the contamination of the matrix material by the pore-former. The mechanical properties and applications of the porous metal dictate the level of contamination permissible in the material. The mechanical properties of titanium and its alloys are greatly reduced by the presence of oxygen, carbon, nitrogen or hydrogen. Many other elements will also serve to compromise titanium's mechanical properties. Additionally, titanium becomes more reactive as temperature increases. In an embodiment where titanium is used as the matrix material, it is desirable to presinter the article below the β transis temperature of 880° C. Above this temperature, the titanium powder will begin to dissolve its surface oxide layer, allowing for the bulk of the powder to become contaminated more readily. This principle can be applied to other material of a reactive nature.

In addition to achieving higher porosities than is possible with other extractable particulate approaches, this approach can allow for more complex shapes to be formed at the same porosity. This is because compacts that would otherwise have inadequate strength to support a cantilevered or top heavy section can have additional strength imparted to them by presintering the compact prior to extraction.

Upon extraction of the pore former, the metal powder matrix structure can be sintered by known means to densify the matrix material. Sintering conditions are determined by the properties of the powder being sintered. The times, temperatures, pressures and atmospheres used in a sintering cycle are selected based on the nature of the material being sintered. Sintering of metal powders is a well understood field and the selection of a sintering cycle can be made by those skilled in the art. Depending on desired properties of the final porous article, the material may be subjected to post sintering processes such as hot isostatic pressing (HIP) to further density the article.

This invention can be applied to powder injection molded (PIM) parts as well as compacted parts. Present PIM approaches use an extractable particulate that can be removed during first stage debinding. The absence of extractable particulates during thermal (second stage) debinding allow capillary forces to pull the porous structure together, creating undesirably high shrinkages. This invention allows for retaining the extractable phase through first stage debinding and presintering the molded particles prior to removing the extractable phase.

In present PIM applications, care must be taken to select particles that have sufficient angularity to resist the capillary forces of the binder that are present during thermal debinding. This is difficult and limits the powders that may be used. By presintering prior to removal of the extractable particulate, less angular, more spherical metal powders may be used. Generally more spherical powders are desirable in PIM applications because they allow for higher loading of the powder in the binder, reducing shrinkage during sintering and minimizing distortion issues.

As described above, the extractable particulate may be left in place and the porous article machined, shaped, and/or finished at a surface thereof before any final sintering is to take place. In this manner, the particulate may protect the integrity of the pores at the surface of the article. That is, the particulate may help prevent the closure or smearing shut of the pores as the article is processed to a completed shape, texture or other physical form.

As indicated, it may be desirable to roughen the surface of the foam or to add a specific texture. For instance, in many orthopedic applications a rough surface is considered desirable on in-growth surfaces. Manipulating the surface texture presents many of the same problems encountered when trying to machine a porous metal material. By machining the article in a “green” or undensified state as indicated, these difficulties can be overcome.

In addition to using green machining to create the final dimensions of an article or part, it can also be used to create a desired surface finish. Most machining methods, especially lathe and mill operations may be used to impart a surface texture by combining the proper cutting tool and tool path.

Since shrinkage may be encountered during a final sintering operation, it may be desirable to form the compact in a oversized tool and affix it to a substrate. When the compact shrinks, the porous material will shrink to the desired dimensions on the substrate.

A thin layer of water soluble wax can be used as an adhesive, and has the advantage of being able to be removed during the water extraction process. The adhesive should be chosen so as to present no contamination issue at the interface between the porous material and the substrate. Additionally, a degree of presintering, as described above may be employed before machining the article. In this manner, the degree of shrinkage that results may be kept to a minimum.

Some mixtures of metal powder and extractable particulates may exhibit “springback” when the pressure of the compacting operation is removed. In this case the compact may be slightly oversized. In this case the tool should be undersized to create a compact of the desired dimensions.

In addition to using the tool to form distinct aspect of the final part, articles may be cut from larger bulk sections of the compacted material. This invention may readily be adapted to other metal by appropriate selection of the extractable material and processing conditions.

The present invention includes articles having variable porosity. In addition to structures having a substantially homogenous porosity, the present invention includes embodiments having non-homogenous porosity such as layers of differing porosity or porous layers on dense substrates.

The forming of articles with non-homogeneous porosity can be accomplished sequentially or simultaneously. An example of a simultaneous route is the die compaction or cold isostatic pressing of multiple layers of material at the same time, where each layer has a pore-former with differing granule properties. This example may also include a layer of metal powder without any extractable material, resulting in a layer of high density. An example of a sequential route is the compaction of a metal powder and extractable particulate mixture onto a solid metal part or substrate. An other example of a sequential route is to take a previously compacted article and compacting a layer onto it. The previously compacted article or the layer may or may not contain an extractable particulate. As used herein, the term layer includes any section of material having properties that differ from those surrounding it.

The co-forming of a porous layer and a non-porous layer has advantages in many applications. The solid layer may allow easy fixation of the porous material to a substrate. The solid layer may also serve as barrier to isolate the porous material. The solid layer can also serve as a mating surface in a thermal management device and the porous material as a high surface area heat dissipating device. The porous material may serve as kinetic energy dissipating element and the solid layer serve to distribute the force of an impact across the porous section.

An article may be formed having layers of differing porosity, providing a part that has a porosity gradient or differing porosity characteristics in an article. By co-forming materials with layers containing varying amounts of extractable particulate, an article having areas of differing porosity can be formed. Included in this approach is the forming of a layer that is essentially free of pores. In this manner an article with porous areas on the outside and dense areas on the inside could be formed. Alternatively, an article that is porous on the inside and dense on the outside may also be formed.

In one example a 1.25″ radius hemispherical mandrel can be used to form a compact having a concave surface of precise dimensions. The compact can be removed from the mandrel after compaction and placed fixed onto a substrate material or left free-standing. If so desired the compact can be green machined at this point to give details, attributes or precision to the compact. The compacted article can then be processed as previously described.

FIG. 4 shows an example of an article with layers of varying porosity manufactured according to the present invention. FIG. 4 is a cross-section of an article having a layered nature with alternating section of differing predetermined porosity. A first region 40 has a 85 volume percent porosity and pore sizes in the range of 200-850 microns, a second region 41 is a dense layer which has been sintered to a closed porosity of approximately 95 percent dense.

The article in FIG. 4 is formed by first adding titanium powder of a −325 mesh to a conventional die compaction tool and compacting the material gently so as to distribute it evenly around the tool without significant densification. Then a mixture of 15 volume percent −325 mesh commercially pure titanium metal powder and 85 volume percent −20 mesh potassium chloride with 0.05 g per cc of mixture PEG 200 as a homogenizing aid is added to the tool and compacted at 38 ksi. The resulting compact has a defined layer of porous material and non-porous material. The compact is removed from the tool and heated in an partial pressure of argon at a rate of 6° C./min to 800° C. and held for 1 hour. After cooling down the extractable phase is removed by immersing the part in hot water, drying the article and sintering in a partial pressure of argon by heating at a rate of 10° C./min to 1255° C. and soaking for two hours. The highly porous region 40 is formed by the mixture of titanium powder and extractable particulate. The second region 41 is of higher density titanium formed by the layer of titanium powder without any extractable particulate.

It is also desirable to form a porous surface onto a preexisting structure or substrate. An example of this is an orthopedic implant. The bulk of the implant is a solid piece that provides the strength of the implant. Porous areas are desired on this piece at areas where bone in-growth is desired. In one example, a mixture of metal powder and extractable particulate is first cold isostatically pressed onto an implant body in the areas where in-growth is desired.

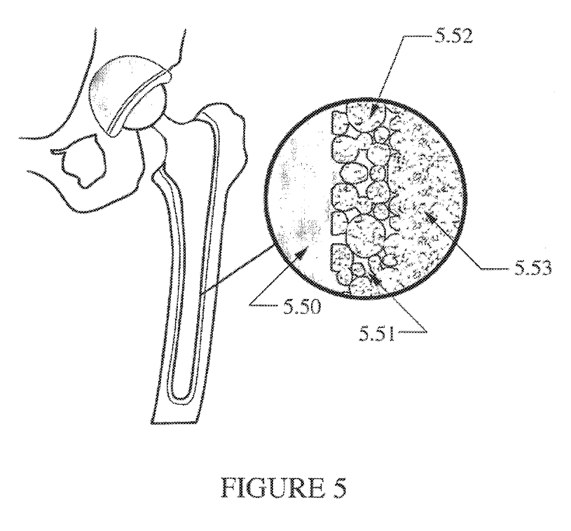

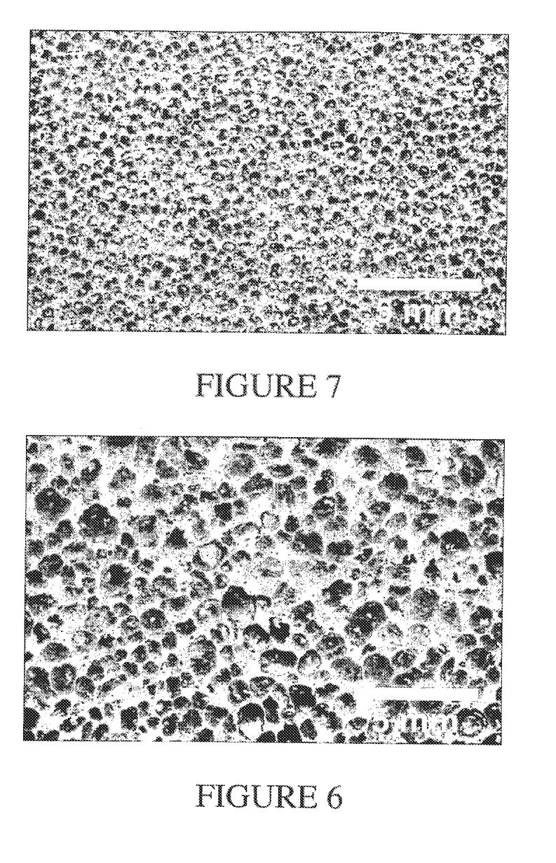

A layered compact made in accordance with the present invention may be used as an orthopedic implant shown in FIG. 5. The solid base 50 metal of the implant has a porous outer surface 51. The in-growth area 52 is the region where bone has grown into the porous surface of the implant. The bone 53 is thereby structurally affixed to the implant. In this example, a compact having layers of porous material and non-porous material is formed by placing the solid stem of the implant into a rubber tool designed to hold it and also allow the mixture of metal powder and extractable particulate to be poured into a gap around the areas on the stem where a porous area is desired. Then a mixture of 20 volume percent −325 mesh commercially pure titanium metal powder and 80 volume percent −20 mesh potassium chloride with 0.05 g per cc of mixture PEG 200 as a homogenizing aid is poured into the tool and tapped to assure proper filling. The tool is then sealed and cold isostatically pressed at 40 ksi. The resulting article has a defined layer of porous material and non-porous material. The compact is removed from the tool and heated in an partial pressure of argon at a rate of 6° C./min to 800° C. and held for 1 hour. After cooling down, the article is placed in a water bath at 40° C. to remove the extractable phase. After the removal is complete the part is dried and sintered in a partial pressure of argon at a heating rate of 10° C./min to 1290° C. and held for 1 hour.

When a layer is compacted onto a substrate the close mechanical interface created allows the matrix material to diffusion bond with the substrate material at elevated temperatures. This can typically be done in the same temperature regime used to sinter the matrix material.

When compacting a layer onto a substrate, the layer does not have to be the same material as the substrate. However the materials should be selected to be compatible with the subsequent processes of sintering the matrix and diffusion bonding the layer to substrate the substrate.

The present invention also includes large porous shapes from which a desired shape can be formed using conventional metal forming methods. This technique of forming a desired porosity can also be applied to many binder assisted forming routes such as powder injection molding, extrusion or casting. The specific nature of the binder and forming route must be considered in the selection of an extractable particulate.

For example, in the field of powder injection molding, the binder used to form the powder article is typically removed in two stages. The first stage is typically an extraction stage where a binder phase is removed by solvent extraction, catalytic decomposition, or evaporation. The second phase of the binder is typically removed by thermal decomposition. In this invention the extractable particulate is retained through the first stage debinding and is left in the part at least until enough presintering has occurred to impart strength the article. In most cases this will be after the second binder phase has been completely removed, but may be when the second phase is only partially removed. After this presintering stage the extractable particulate is removed and then article is sintered to the desired density.

Powder injection molding compounds have two components, the powder system and the binder system. The powder system typically contains the powders that are to be formed and sintered. The powder system does not melt or undergo and significant changes during the injection molding stage. The binder system is typically composed of various polymers and waxes and melts to allow the forming of the desired shape. In the discussion of powder injection molding, the extractable particulate is added to the powder system since it is this system that is being modified to create the desired porosity.

In one embodiment, the use of an extractable particulate can be combined with commercially available binders. For example, a commercially pure titanium article having 70 percent porosity can be formed by using powder system of the composition: 70 volume % Potassium chloride and 30 volume % Titanium powder (−45 microns).

This powder system can be combined with a commercially available water, acetone or alcohol soluble binder composition or system such as F566 Binder System manufactured by Praxis Technology, Queensbury, N.Y. The final compound would have a composition of 85 percent by weight powder (extractable and matrix forming) and 15 percent binder. The mixing of the materials may be done sequentially to allow proper mixing of the metal powders and the binder prior to adding the extractable particulate. For example, with the above powder system, the titanium powder is combined with binder system and mixed until the binder system has melted and a homogeneous mixture achieved. Following the mixture of the metal powders and the binder the extractable particulate can be added and mixed until homogeneous.

The necessity for sequential mixing is dictated by the specific binder system used. For instance, the binder system may incorporate emulsified surfactants which contain water that is evaporated off during mixing. If the extractable particulate is water soluble, it should be added after the water has been evolved from the mixture.

In one example, after the feedstock is mixed, it is injection molded to form the article. The injection molding conditions are based upon the specific binder system used. In this example the melt temperature would be 180-190° C. and the molding pressure about 800 psi. After the article is formed it is immersed in warm acetone at 40-45° C. The use of acetone allows the removal of the extractable phase of the binder without removing the extractable particulate. The length of immersion is based upon the size of the molded part. It is helpful to add clean acetone and removed used acetone from the bath to maintain an advantageous concentration gradient between the internal section of the part and the water bath. After removal from the water bath the compact is dried to remove any remaining acetone. Following this the article is thermally debound by heating at a rate of 4° C. min to 340° C. and holding for 1 hour. The article is then heated at a rate of 8° C./min to 1000° C. and presintered for 1 hour. Both the debinding and the presintering are done under an argon atmosphere. The article is then cooled to room temperature and the extractable particulate (potassium chloride) is removed by placing the article in a water bath at 30-60° C. After the bulk of the potassium chloride has been removed, the water is replaced several times to remove any traces of the extractable particulate. In practice a conductivity meter can be used to determine the completeness of the removal and rinsing of the potassium chloride. After removal from the water bath the compact is dried to remove any remaining water. This can be done in air at 20-60° C. The article is heated at a rate of 10°/min in argon to 1300° C. for 1 hour.

An article having a porous and non-porous section can be produced via PIM. This is achieved by co-molding the article from two feedstocks. The first of these feedstocks is a conventional PIM feedstock containing a binder and a metal powder. The second feedstock contains a binder, a metal powder and an extractable particulate. The first feedstock is used to form the section of the article intended to be non-porous. The second feedstock is co-molded onto the article to form the area intended to be porous.

In an example of forming an article composed of ASTM F-75 having porous and non-porous sections via PIM, the conventional feedstock used to form the non-porous region is prepared using a commercially available water, acetone or alcohol soluble binder system such as F566 Binder System manufactured by Praxis Technology, Queensbury, N.Y. This feedstock is compounded using the following composition: 66 volume % ASTM F-75 metal powder, −20 microns and 34 volume % F566 Binder System.

The feedstock used to form the porous region of the article is compounded using the following composition: 44 volume % potassium chloride and 22 volume % ASTM F-75 metal powder (−20 microns) and 34 volume % F566 Binder System.

When preparing the above feedstock system, the metal powder is combined with binder system and mixed until the binder system has melted and a homogeneous mixture achieved. Following the mixture of the metal powders and the binder the extractable particulate (potassium chloride) can be added and mixed until homogeneous.

After the feedstocks are mixed, they are injection molded to form the article. In this example the melt temperature would be 180-190° C. and the molding pressure about 800 psi. for both materials. In this example the non-porous feedstock is molded first and the porous material is molded onto it afterwards. During molding of the feedstock containing the extractable particulate, binder and metal powder rich areas may form on the outer surface of the part. These areas can be removed by grit blasting, machining or other removal methods. After the article is formed it is immersed in warm acetone at 40-45° C. The use of acetone allows the removal of the extractable phase of the binder without removing the extractable particulate. The length of immersion is based upon the size of the molded part. It is helpful to add clean acetone and removed used acetone from the bath to maintain an advantageous concentration gradient between the internal section of the part and the water bath. After removal from the water bath the compact is dried to remove any remaining acetone. Following this the article is thermally debound by heating at a rate of 4° C. min to 340° C. and holding for one hour. The article is then heated at a rate of 8° C./min to 1000° C. and presintered for one hour. Both the debinding and the presintering are done under an argon atmosphere. The article is then cooled to room temperature and the extractable particulate (potassium chloride) is removed by placing the article in a water bath at 30-60° C. After the bulk of the potassium chloride has been removed, the water is replaced several times to remove any traces of the extractable particulate. In practice a conductivity meter can be used to determine the completeness of the removal and rinsing of the potassium chloride. After removal from the water bath the compact is dried to remove any remaining water. This can be done in air at 20-60° C. The article is heated at a rate of 10°/min in argon to 1325° C. for one hour.

The properties of the porous metal described above provide a strong bone fixation device that may include a threaded element. A surface with a properly tailored porosity will allow the bone to grow into the surface, creating a stronger interface between the bone and the device and reducing the ability for the fixation device to loosen. Additionally upon installation, there is compressive engagement between the bone and the in-growth material at the threads. Since the device is adequately immobile relative to the bone, rapid osteointegration onto and into the porous metal takes place.

Porous bone fixation devices can be manufactured as described above by forming a composite article comprising a porous section and a non-porous section. To allow the shaping of orthopedic devices having both a homogeneous porous section and a non-porous section, in one embodiment the porous section is first formed as provided herein and then the non-porous section is formed. The porous section can be formed by several different methods of consolidating a powder mixture into a shape. These include by way of example, preparing a metal powder mixture as discussed herein for either powder particle deformation or binder assisted forming processes, and then shaping the metal powder mixture by injecting molding, die compaction, or isostatic pressing. Once the porous section is formed, the non-porous section can be formed by, for example, PIM. The porous and non-porous sections form a composite article and can be further processed by green machining and sintering.

The techniques of forming porous articles via pressing can be combined with PIM to co-form complex geometries with porous and dense sections. In a preferred embodiment, a bone screw with threads that are at least partially porous is formed by injection molding the dense portion of the screw into a mold containing a previously formed green article. This green article or “preform” includes the pore former and metal powder matrix, and has been pressed to give it strength for handling and machining. In one example, a feedstock of 65 volume percent pore former and 35 volume percent titanium metal powder is cold isostatically pressed into a cylindrical shape around a mandrel that forms the inner diameter of the cylinder. Because it is formed on a mandrel the inner diameter is precisely shaped and can be used to fixture the cylinder during a green machining operation. The preform cylinder is then turned on a lathe to precisely fit an insert cavity in an injection molding tool. The injection molding tool is shaped to form the nonporous section of the device in a complementary fashion with the preform. Metal injection molding feedstock is injected into the mold forming the non-porous section of the device. The non-porous section and porous section comprise a composite article. The green composite article is removed from the mold, and a lathe can be used to cut, a thread into the composite article. Green machining the threads after injection molding allows for versatility of the machined geometry; for instance, very thin sections can be machined into the porous section because it is supported by the metal injection molded feedstock forming the non-porous section.

In the context of the previous example, the porous preform can be an open or closed end cylinder. A closed end cylinder creates a continuous porous section at the tip of the screw. An open ended cylinder allows the injection molded feedstock to continue past the porous section and form another external dense section, for example a screw tip or shank. The metal injection molded material is also used to form parts of the geometry not specifically related to the threaded section, such as the head, or portion of the screw used for engaging the device by a tool used during orthopedic surgical procedure.

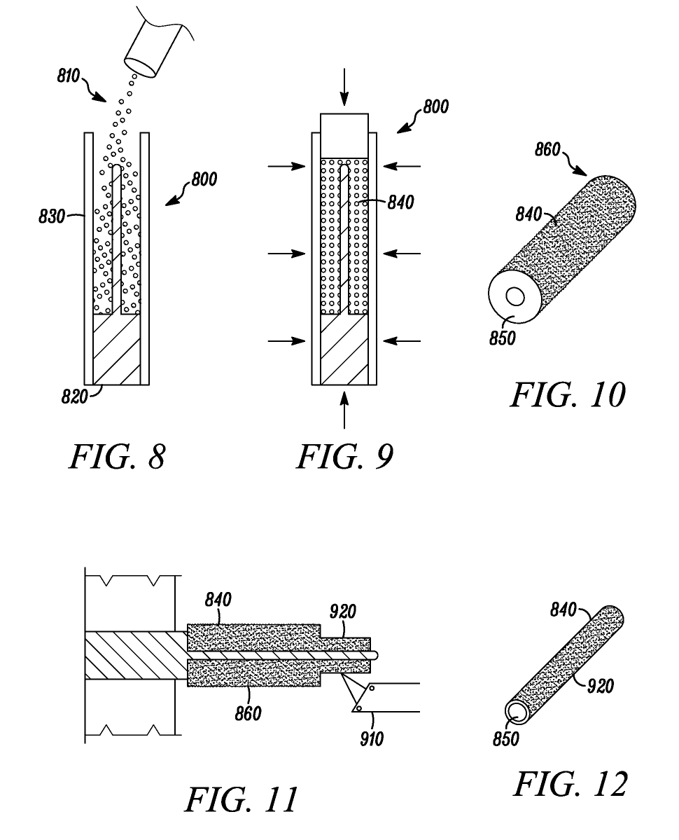

FIGS. 8-16 illustrate an embodiment of forming a porous bone fixation device. FIG. 8 shows the mixture of metal powder and pore former 810 used to form the porous section being poured into an isostatic tool 800 for isostatic pressing. The tool consists of a steel mandrel 820 which forms the inner diameter of the cylindrical porous perform and as well as one end of the cylinder and the rubber outer sleeve 830 of the tool that holds the feedstock around the mandrel and allows the transmission of force to the feedstock after the tool is sealed with a rubber plug (not shown). Referring now to FIG. 9, the tool 800 having been filled and sealed is exposed to isostatic pressure to form a pressed cylinder 840. FIG. 10 is an embodiment of the pressed cylinder 840. After it has been removed from the tool 800 it has a precisely defined inner opening 850 and a round but less precise outer surface 860.

FIG. 11 illustrates an embodiment of machining the pressed cylinder 840 on a lathe with a cutting tool 910. The outer surface 860 of the pressed cylinder 840 is machined to a smooth surface 920 having a predetermined diameter. FIG. 12 is an embodiment of a machined porous cylinder 840 now having a precise outer diameter 920 and an inner opening 850.

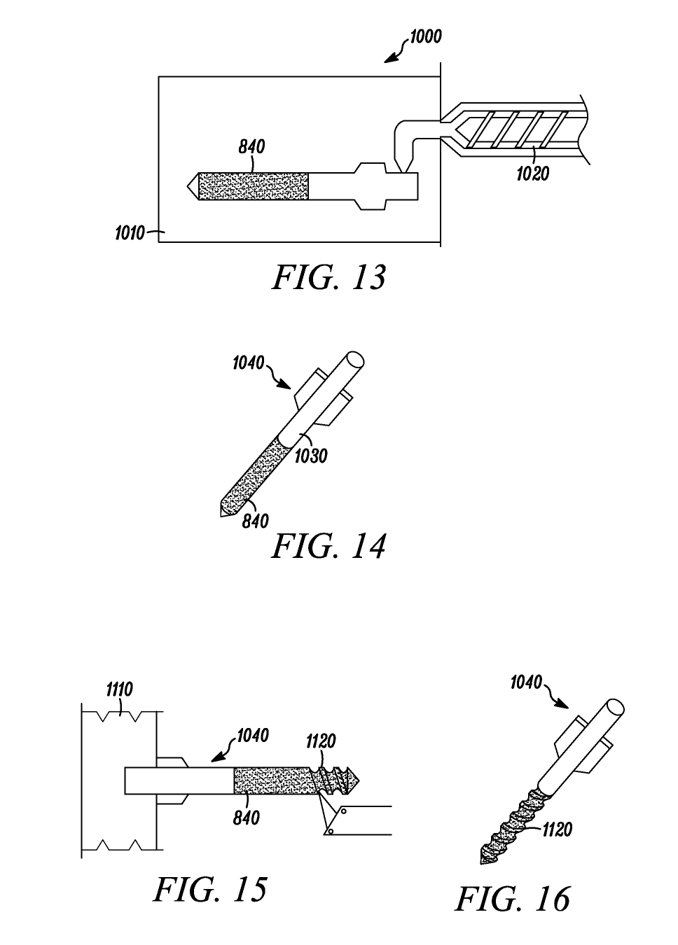

FIG. 13 illustrates an embodiment of forming a porous bone fixation device in an insert molding machine 1000. The machined pressed cylinder 840 is placed in an injection molding tool 1010 and metal injection molding feedstock is injected from an injection device 1020 into the tool 1010 and through the inner opening 850 of the pressed cylinder 840 (not shown). FIG. 14 is an embodiment of a composite article 1040 consisting of the machined pressed cylinder 840 and a injection molded feedstock 1030 after injection molding. It will be understood that many details such as the tip geometry and the geometry of the head of the screw can be formed in the injection molding operation.

FIG. 15 illustrates an embodiment of forming a composite article 1040 by mounting it on a lathe 1110 and machining threads 1120 into the pressed cylinder 840. Referring to FIG. 16, an embodiment of a composite article 1040 having porous threads 1120. The composite article 1040 can be further processed by removing the pore former and extractable phase of the metal injection molding binder and debinding and sintering the article as described herein.

The metal injection molded material typically needs to undergo an extraction of some or all of the binder phases prior to sintering. As in the case where both the porous and dense section are injection molded, it is preferred that the pore former and the extractable binder phase be removed in the same step. This is accomplished by selecting the pore former and the removable binder phase to be soluble in the same solvent, as described herein. For example, a potassium chloride pore former is combined with a water soluble metal injection molding feedstock which uses polyethylene glycol as a removable phase.

Screw-type geometries are one embodiment and many variations with porous and dense sections are also contemplated. For example, orthopedic implants such as femoral knees and acetabular cups are other embodiments. Porous preforms are shaped into the desired shape and placed in an injection molding tool. Metal injection molding feedstock is then injected against or around the preform to form a co-formed green article. After molding, the article can be further green machined if desired and processed by removing the pore former and sintering the article. The process is very versatile and can be used to form devices having both porous and dense sections.

The function of a bone fixation device is greatly enhanced by providing a porous in-growth area along the threaded section of the fixation device. Various configurations are possible depending on the desired application. For example, the entire thread form may be porous, or just the thread or just the land on the minor diameter between them may be porous.

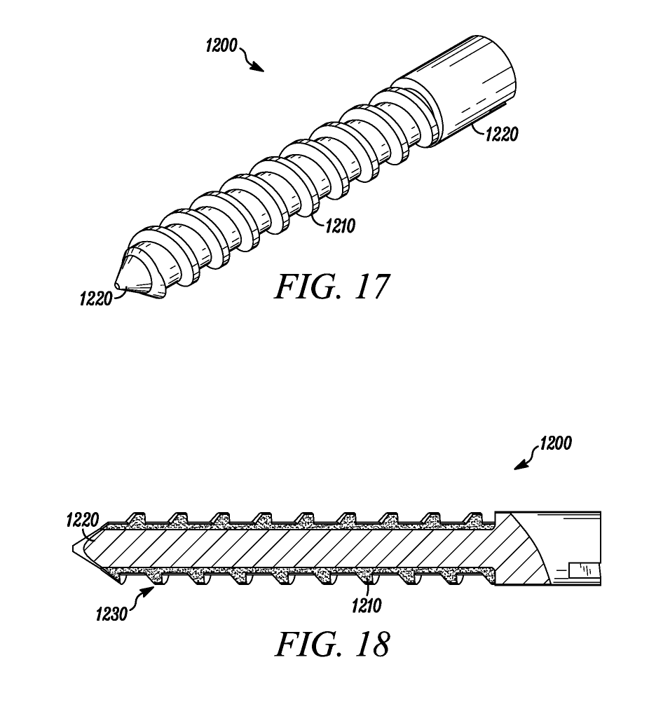

Bone fixation devices with porous threads such as those shown in FIGS. 17-26 with a porosity of 65% and average inter-connecting pore size of 60 microns have demonstrated shear strength of a minimum of 20 Megapascal and up to or exceeding 40 Megapascal. Many other variations of the thread form are possible. For instance, if greater thread strength is desired, a double helix type thread could be used, with one thread being porous and the other being dense.

Referring to FIG. 17, a bone fixation device 1200 has a porous titanium threaded section 1210 and a metal shaft 1220 along the length of the device. FIG. 18 shows the device 1200 of FIG. 17 in partial cross-section, including the porous titanium threads 1210 and the metal shaft 1220. In this embodiment, the major diameter of the thread has a land 1230.

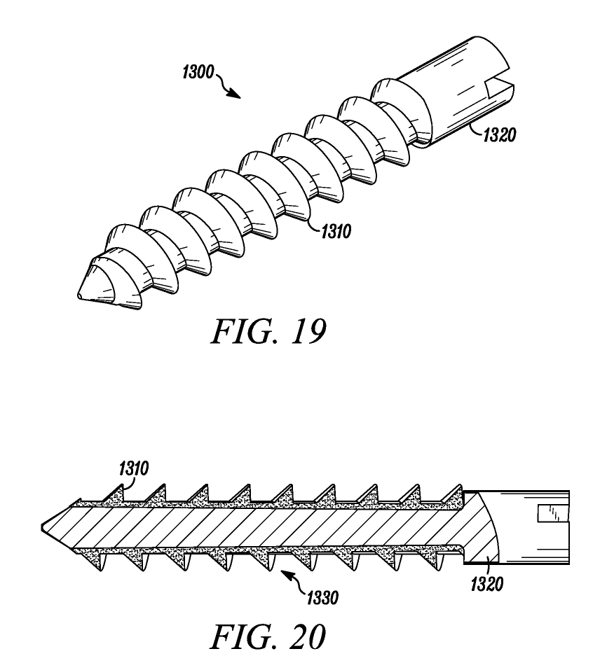

Referring to the embodiment shown in FIGS. 19-20, a bone fixation device 1300 has a porous threaded section 1310 and a dense metal shaft 1320 along the length of the device. In this embodiment the threaded section 1310 comes to a sharp point at the major diameter to form a crest 1330.

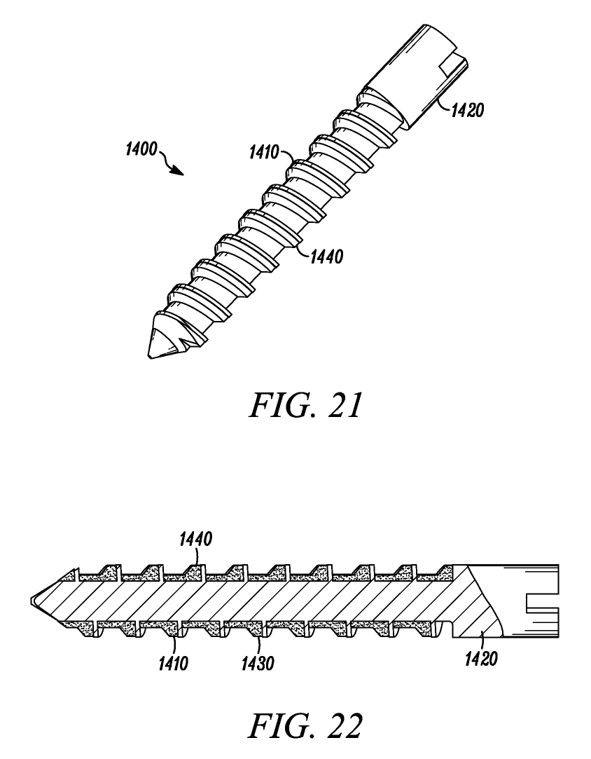

Turning now to FIGS. 21-22, another embodiment of a bone fixation device 1400 has with a portion of the thread 1440 that is non-porous. This adds strength to the edge of the thread. This also provides a solid surface to bear load during insertion. As can be seen in the illustrated cross-section of device 1400 in FIG. 22, the device 1400 has threads with a non-porous section 1440 and a porous section 1410 around a shaft 1420. The major diameter of the thread has a land 1430 rather than a crest edge in this embodiment.

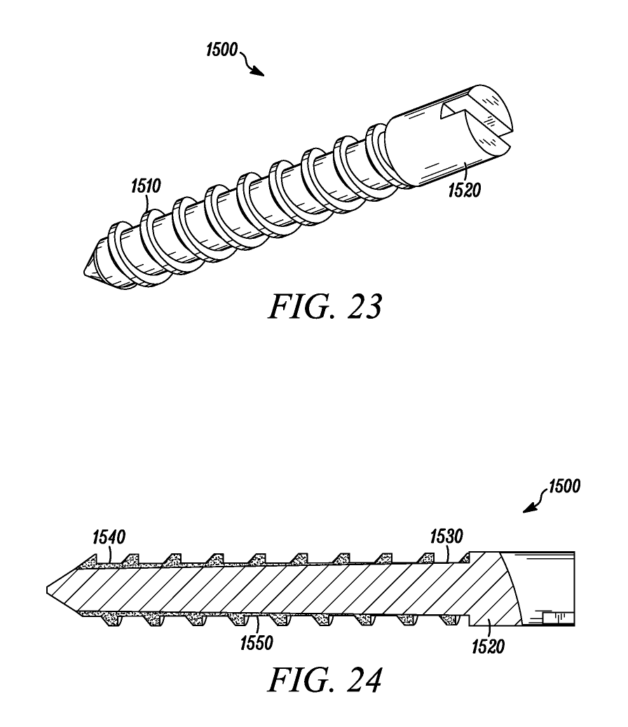

Referring to the embodiment in FIGS. 23-24, a bone fixation device 1500 has porous threads 1510 with the minor diameter of varying thickness of porous material over a tapered metal shaft 1520 along the length of the device. As can be seen in the illustrated cross-section of the device 1500 in FIG. 24, the porous material at the minor diameter near the tip of the device in region 1540 is thicker and gradually transitions along the metal shaft 1520 to a thinner porous region 1550. No porous material is located at the minor diameter near the shoulder of the device in region 1530.

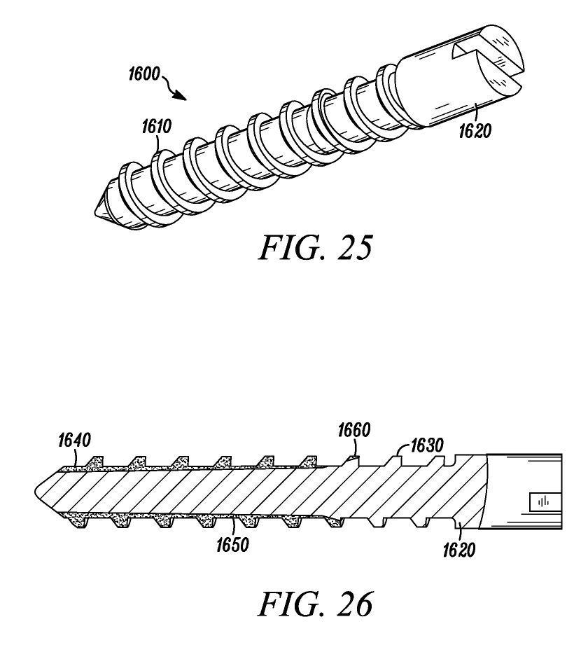

Referring to FIGS. 25-26, a bone fixation device 1600 has a porous threaded section 1610 near the tip of the device, a non-porous threaded section 1630 near the shoulder of the device, and a tapered metal shaft 1620 along the length of the device. As can be seen in the illustrated cross-section of the device 1600 in FIG. 26, the porous material at the minor diameter near the tip of the device in region 1640 is thicker and gradually transitions along the metal shaft 1620 to a thinner porous region 1650. Further along the metal shaft 1620 towards the shoulder, the threads in region 1660 are partially porous and partially non-porous. Neither the minor diameter of the device nor the threads include a porous material near the shoulder of the device in section 1630.

While FIGS. 17-26 are illustrated examples of porous bone fixation devices, it will be understood that many variations are possible including but not limited to bone anchors and dental implants. In addition, these examples do not detail the head of the bone fixation device because it is understood the head can manifest itself in many differing configuration or designs. While the examples depict fixation devices with dense tips, the tips could also have a porous coating or section on them. Similarly, the location of the porous section of a bone fixation device is a design choice enabled hereby. While this invention is described as having a preferred design, the present invention can be further modified with in the spirit and scope of this disclosure.

The strength of the porous threads of a bone fixation device is critical to ensure the success of the implant in the animal or human body. An embodiment of a sintered titanium bone screw with porous threads was used for testing. This bone screw had major diameter of 6.5 mm a minor diameter of 5.1 mm, an overall length of 57 mm, on major thread land of 1 mm, a thread length of 26 mm and a pitch of 2.7 mm. The bone screw was inserted a fixture at a depth of between 13-23 mm (0.5-0.9 in) to test the axial pull-out strength. The tool and fixation device were mounted in a tensile testing machine and tested until thread failure. A cross head speed of 0.13 cm/min (0.05 in/min) was used. The average axial pull out force was 7830 N (1760 lbf). The normalized shear strength based on surface area in the shear plane is at least 40 MPa (5800 psi).

To test the integrity of the porous thread as well as the interface between the porous material and the dense portion, titanium fixation devices with porous threads were mounted in a steel tool with a hole that was threaded to match the porous fixation device. The fixation device was subjected to cyclic axial loading having an amplitude of 2,200 N (500 lbf). After 10 million cycles the porous section was intact without failure or deterioration of the thread profile.

The nature of this invention can be applied to binder systems used in the binder assisted forming of sinterable powders. The field of binder assisted forming encompasses many different forming techniques such as the injection molding, compression molding, compaction, extrusion, or green machining of articles comprised of a powder and binder mixture.

Although the present invention has been described in terms of examples and presently preferred embodiments, it is to be understood that such disclosure is not to be interpreted as limiting. Various alterations and modifications will no doubt become apparent to those skilled in the art after having read the above disclosure. Accordingly, it is intended that the claims be interpreted as covering all alterations and modifications as fall within the true spirit and scope of the invention.

What is claimed is:

1. A bone fixation device, comprising:
a head portion;
a metal shaft defining a length, the metal shaft extending from the head portion; and
a porous portion extending along the length having a predetermined porosity defined by an extractable material.

2. The device of claim 1, wherein the metal shaft comprises a metal selected from the group consisting in titanium and tantalum.

3. The device of claim 1, wherein the porous portion has a thread defining a major diameter and a minor diameter.

4. The device of claim 1, wherein a substantially non-porous portion extends along the length.

5. The device of claim 3, wherein the thread has a crest at the major diameter.

6. The device of claim 3, wherein the thread has a flat land at the major diameter.

7. The device of claim 4, wherein the non-porous portion has a second thread.

8. The device of claim 3, wherein the porous portion has a thickness at the minor diameter that is increasing along the length extending from the head portion.

9. An implant assembly for providing fixation to a bone comprising:
a head portion;
a metal shaft defining a length, the metal shaft extending from the head portion; and
a porous portion extending along the length and having a thread defining a major diameter and a minor diameter, wherein the implant assembly has a shear strength of greater than 20 Megapascal.

10. The implant assembly of claim 9, wherein a substantially non-porous portion having a second thread extends along the length.

11. The device of claim 10, wherein the metal shaft comprises metal selected from the group consisting in titanium and tantalum.

12. The device of claim 9, wherein the thread has a crest at the major diameter.

13. The device of claim 9, wherein the thread has a flat land at the major diameter.

14. The device of claim 9, wherein the porous portion has a thickness at the minor diameter that is increasing along the length extending from the head portion.

15. The device of claim 14, wherein the major diameter and the minor diameter are substantially the same along the length.

16. An implant assembly for providing fixation to a bone comprising:
a head portion;
a metal shaft defining a length, the metal shaft extending from the head portion; and
a porous thread extending along the metal shaft defining a major diameter and a minor diameter, wherein the implant assembly has a shear fatigue life of at least 10,000,000 cycles.

17. The implant assembly of claim 16, wherein a substantially non-porous thread extends along the length.

18. The device of claim 17, wherein the metal shaft comprises metal selected from the group consisting in titanium and tantalum.

19. The device of claim 16, wherein the porous thread has a crest at the major diameter.

20. The device of claim 16, wherein the porous thread has a flat land at the major diameter.

21. The device of claim 16, wherein the porous thread has a thickness at the minor diameter that is increasing along the length extending from the head portion.

22. The device of claim 22, wherein the major diameter and the minor diameter are substantially the same along the length.

23. A method of making a porous implant device, comprising the steps of:
blending an extractable particulate and a first powder of metal particles to form a homogeneous mixture;
forming an article comprising the mixture;
providing the article in a mold;
injecting a feedstock into the mold comprising a second powder of metal particles to form a composite article;
removing the extractable particulate to form pores in the composite article; and
sintering the composite article.

24. The method according to claim 23, further comprising machining the composite article prior to sintering.

25. The method according to claim 23, further comprising forming a thread on the composite article prior to sintering.

26. The method according to claim 23, wherein the removing step comprises removing a binder from the feedstock.

27. The method according to claim 23, wherein the forming step comprises compacting the first powder of metal particles.

28. The method according to claim 23, wherein the forming step comprises adding a binder to the first powder of metal particles.

29. The method according to claim 27, wherein the compacting comprises deforming the metal particles.

30. The method according to claim 25, wherein the thread comprises a crest.

31. The method according to claim 25, wherein the thread comprises a flat land.

32. The method according to claim 23, wherein the article comprising the mixture is in the shape of a cylinder.

33. The method according to claim 23, wherein the composite article is selected from the group consisting of a bone screw, bone anchor, dental anchor, and orthopedic implant.
